(19) United States
(12) Patent Application Publication
(10) Pub. No.: US 2008/0200436 A1
(43) Pub. Date: Aug. 21, 2008

(54) Combination Comprising Zd6474 And An Antiandrogen

(76) Inventor: Stephen Robert Wedge, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/666,762

(22) PCT Filed: Nov. 1, 2005
(86) PCT No.: PCT/GB05/04220

§ 371 Date: Dec. 21, 2007

(30) Foreign Application Priority Data
Nov. 3, 2004 (GB) 0424339.0

(51) Int. Cl. A61K 31/675; A61K 31/506; A61P 43/00

(52) U.S. Cl. 514/90; 514/266.22

(57) ABSTRACT

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour, which comprises the administration of ZD6474 in combination with androgen ablation; to a pharmaceutical composition comprising ZD6474 and an antiandrogen; to a combination product comprising ZD6474 and an antiandrogen for use in a method of treatment of a human or animal body by therapy; to a kit comprising ZD6474 and an antiandrogen; to the use of ZD6474 and an antiandrogen in the manufacture of a medicament for use in the production of an antiangiogenic and/or vascular permeability reducing effect in a warm-blooded animal such as a human which is optionally being treated with ionising radiation.

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour, which comprises the administration of ZD6474 in combination with androgen ablation; to a pharmaceutical composition comprising ZD6474 and an antiandrogen; to a combination product comprising ZD6474 and an antiandrogen for use in a method of treatment of a human or animal body by therapy; to a kit comprising ZD6474 and an antiandrogen; to the use of ZD6474 and an antiandrogen in the manufacture of a medicament for use in the production of an antiangiogenic and/or vascular permeability reducing effect in a warm-blooded animal such as a human which is optionally being treated with ionising radiation.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fins-like tyrosine kinase receptor, Flt-1 (also referred to as VEGFR-1), the kinase insert domain-containing receptor, KDR (also referred to as VEGFR-2 or Flk-1), and another fins-like tyrosine kinase receptor, Flt-4. Two of these related RTKs, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington D.C.), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J., Orci, L. and Vassalli, J. D., Enzyme Protein, 49: 138-162, 1996.). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Buittner, M., Rziha, H-J., and Dehio, C., EMBO J., 18: 363-374, 1999; Zeng, H., Sanyal, S. and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

Quinazoline derivatives which are inhibitors of VEGF receptor tyrosine kinase are described in International Patent Applications Publication Nos. WO 98/13354 and WO 01/32651. In WO 98/13354 and WO 01/32651 compounds are described which possess activity against VEGF receptor tyrosine kinase (VEGF RTK) whilst possessing some activity against epidermal growth factor (EGF) receptor tyrosine kinase (EGF RTK). ZD6474 is 4-(4-bromo-2-fluoroanilino)-6-methoxy-7-(1-methylpiperidin-4-ylmethoxy)quinazoline:

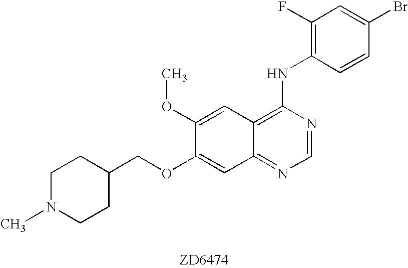

ZD6474 falls within the broad general disclosure of WO 98/13354 and is exemplified in WO 01/32651. ZD6474 is a potent inhibitor of VEGF RTK and also has some activity against EGF RTK. ZD6474 has been shown to elicit broad-spectrum anti-tumour activity in a range of models following once-daily oral administration (Wedge S R, Ogilvie D J, Dukes M, et al. ZD6474 inhibits vascular endothelial growth factor signaling, angiogenesis, and tumour growth following oral administration. Cancer Res 2002;62:4645-4655).

In WO 98/13354 and WO 01/32651 it is stated that compounds of their inventions: “may be applied as a sole therapy or may involve, in addition to a compound of the invention, one or more other substances and/or treatments. Such conjoint treatment may be achieved by way of the simultaneous, sequential or separate administration of the individual components of the treatment.”

WO 98/13354 and WO 01/32651 then go on to describe examples of such conjoint treatment including surgery, radiotherapy and various types of chemotherapeutic agent including “antiandrogens (for example flutamide, nilutamide, bicalutamide, cyproterone acetate), LHRH agonists and antagonists (for example goserelin acetate, luprolide, abarelix), inhibitors of testosterone 5α-reductase (for example finasteride)”.

However nowhere in WO 98/13354 and WO 01/32651 is the specific combination of ZD6474 and androgen ablation suggested.

Nowhere in WO 98/13354 and WO 01/32651 does it state that use of any compound of the invention therein with other treatments will produce surprisingly beneficial effects.

The use of EGF receptor tyrosine kinase inhibitors in combination with antiandrogens is described in International Patent Application No. WO 01/76586.

Androgen ablation may be achieved by surgical and/or chemical means. Surgical castration involves the removal of the testes by surgery. After surgical castration androgens will continue to be produced by the adrenal glands so although the levels of androgens are reduced they are not completely removed. Chemical castration can be achieved by administering an antiandrogen. Antiandrogens can inhibit the effects of androgens produced by the testes and by the adrenal glands so the degree of androgen ablation achieved by chemical castration can be greater than that achieved by surgical castration. Surgical castration and chemical castration can be used together. Examples of antiandrogens include luteinising hormone releasing hormone (LHRH) agonists such as goserelin, buserelin, triptorelin or leuprorelin, LHRH antagonists, non-steroidal antiandrogens such as bicalutamide (or an enantiomer thereof), flutamide and nilutamide and steroidal antiandrogens such as cyproterone acetate and megestrol acetate. The properties and usefulness of some of these antiandrogens have been reviewed, for example in the following documents which are incorporated herein by way of reference

| | |
|---|---|
| bicalutamide | B J A Furr et al., Urology, 1996, 47 (Suppl. 1A), 13-25, |
| | G J C Kolvenbag et al., Urology, 1996, 47 (Suppl. 1A), |
| | 70-79 and European Patent Application No. 0100172 |
| | as the 8th compound listed in the table in Example 6; |
| flutamide | R O Neri, J. Drug Develop., 1987, 1 (Suppl.), 5-9 and |
| | Urology, 1989, 34 (Suppl. 4), 19-21 and United Kingdom |
| | Patent Application No. 1360001; |
| nilutamide | M G Harris et al., Drugs and Aging, 1993, 3, 9-25 and |
| | United Kingdom Patent Application No. 1518444. |

Androgen ablation is frequently used to treat prostate cancer. However in prostate cancer, the benefits of androgen ablation are generally temporary due to the eventual transformation of prostate cancer cells from a hormone-dependent state into a hormone-independent state and/or the clonal selection of androgen-independent prostate cancer cells. It is to be understood that any reference herein to the inhibition of the transformation of prostate cancer cells from a hormone-dependent state into a hormone-independent state is to be taken as equivalent to a reference to the inhibition of the clonal selection of androgen-independent prostate cancer cells.

Unexpectedly and surprisingly we have now found that the particular compound ZD6474 used in combination with a particular selection from the broad description of combination therapies listed in WO 98/13354 and WO 01/32651, namely with androgen ablation, produces significantly better effects than any one of ZD6474 and androgen ablation used alone. In particular, ZD6474 used in combination with androgen ablation produces significantly better anti-cancer effects, particularly significantly better effects on solid tumours than any one of ZD6474 and androgen ablation used alone. More particularly ZD6474 used in combination with androgen ablation produces significantly better anti-cancer effects in prostate cancer.

Anti-cancer effects of a method of treatment of the present invention include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a method of treatment of the present invention is administered to a warm-blooded animal such as a human, in need of treatment for cancer, with or without a solid tumour, said method of treatment will produce an effect, as measured by, for example, one or more of: the extent of the anti-tumour effect, the response rate, the time to disease progression and the survival rate. Anti-cancer effects include prophylactic treatment as well as treatment of existing disease.

According to the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of androgen ablation.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of androgen ablation.

In particular the cancer is prostate cancer.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of androgen ablation.

In particular the cancer involving a solid tumour is prostate cancer.

According to a further aspect of the present invention there is provided a method for inhibiting the transformation of cancerous cells in the prostate from a hormone-dependent state into a hormone-independent state in a warm-blooded animal such as a human, which comprises administering to said animal an effective amount of ZD6474 or a pharmaceutically acceptable salt thereof, before, after or simultaneously with an effective amount of androgen ablation.

According to a further aspect of the present invention there is provided a method for inhibiting the transformation of prostate cells into cancerous cells in a warm-blooded animal such as a human, which comprises administering to said animal an effective amount of ZD6474 or a pharmaceutically acceptable salt thereof, before, after or simultaneously with an effective amount of androgen ablation.

According to a further aspect of the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of an antiandrogen, wherein ZD6474 and an antiandrogen may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of an antiandrogen, wherein ZD6474 and an antiandrogen may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of an antiandrogen, wherein ZD6474 and an antiandrogen may each optionally be administered together with a pharmaceutically acceptable excipient or carrier.

According to a further aspect of the present invention there is provided a method for for inhibiting the transformation of cancerous cells in the prostate from a hormone-dependent state into a hormone-independent state in a warm-blooded animal such as a human, which comprises administering to said animal an effective amount of ZD6474 or a pharmaceutically acceptable salt thereof, before, after or simultaneously with an effective amount of an antiandrogen, wherein ZD6474 and an antiandrogen may each optionally be administered together with a pharmaceutically acceptable excipient or carrier.

According to a further aspect of the present invention there is provided a method for inhibiting the transformation of prostate cells into cancerous cells in a warm-blooded animal such as a human, which comprises administering to said animal an effective amount of ZD6474 or a pharmaceutically acceptable salt thereof, before, after or simultaneously with an effective amount of an antiandrogen, wherein ZD6474 and an antiandrogen may each optionally be administered together with a pharmaceutically acceptable excipient or carrier.

According to a further aspect of the invention there is provided a pharmaceutical composition which comprises ZD6474 or a pharmaceutically acceptable salt thereof, and an antiandrogen, in association with a pharmaceutically acceptable excipient or carrier.

According to a further aspect of the present invention there is provided a combination product comprising ZD6474 or a pharmaceutically acceptable salt thereof and an antiandrogen, for use in a method of treatment of a human or animal body by therapy.

According to a further aspect of the present invention there is provided a kit comprising ZD6474 or a pharmaceutically acceptable salt thereof, and an antiandrogen.

According to a further aspect of the present invention there is provided a kit comprising:
a) ZD6474 or a pharmaceutically acceptable salt thereof in a first unit dosage form;
b) an antiandrogen in a second unit dosage form; and
c) container means for containing said first and second dosage forms.

According to a further aspect of the present invention there is provided a kit comprising:
a) ZD6474 or a pharmaceutically acceptable salt thereof, together with a pharmaceutically acceptable excipient or carrier, in a first unit dosage form;
b) an antiandrogen together with a pharmaceutically acceptable excipient or carrier, in a second unit dosage form; and
c) container means for containing said first and second dosage forms.

According to a further aspect of the present invention there is provided the use of ZD6474 or a pharmaceutically acceptable salt thereof and an antiandrogen in the manufacture of a medicament for use in the production of an antiangiogenic and/or vascular permeability reducing effect in a warm-blooded animal such as a human.

According to a further aspect of the present invention there is provided the use of ZD6474 or a pharmaceutically acceptable salt thereof and an antiandrogen in the manufacture of a medicament for use in the production of an anti-cancer effect in a warm-blooded animal such as a human.

In particular the cancer is prostate cancer.

According to a further aspect of the present invention there is provided the use of ZD6474 or a pharmaceutically acceptable salt thereof and an antiandrogen in the manufacture of a medicament for use in the production of an anti-tumour effect in a warm-blooded animal such as a human.

In particular the tumour is a prostate cancer tumour.

According to a further aspect of the present invention there is provided the use of ZD6474 or a pharmaceutically acceptable salt thereof and an antiandrogen in the manufacture of a medicament for use in inhibiting the transformation of cancerous cells in the prostate from a hormone-dependent state into a hormone-independent state in a warm-blooded animal such as a human.

According to a further aspect of the present invention there is provided the use of ZD6474 or a pharmaceutically acceptable salt thereof and an antiandrogen in the manufacture of a medicament for use in inhibiting the transformation of prostate cells into cancerous cells in a warm-blooded animal such as a human.

According to a further aspect of the present invention there is provided the use of ZD6474 or a pharmaceutically acceptable salt thereof in the manufacture of a medicament for use in the production of an antiangiogenic and/or vascular permeability reducing effect in a warm-blooded animal such as a human which is being treated with androgen ablation.

According to a further aspect of the present invention there is provided the use of ZD6474 or a pharmaceutically acceptable salt thereof in the manufacture of a medicament for use in the production of an anti-cancer effect in a warm-blooded animal such as a human which is being treated with androgen ablation.

In particular the cancer is prostate cancer.

According to a further aspect of the present invention there is provided the use of ZD6474 or a pharmaceutically acceptable salt thereof in the manufacture of a medicament for use in the production of an anti-tumour effect in a warm-blooded animal such as a human which is being treated with androgen ablation.

In particular the tumour is a prostate cancer tumour.

According to a further aspect of the present invention there is provided the use of ZD6474 or a pharmaceutically acceptable salt thereof in the manufacture of a medicament for use in inhibiting the transformation of cancerous cells in the prostate from a hormone-dependent state into a hormone-independent state in a warm-blooded animal such as a human which is being treated with androgen ablation.

According to a further aspect of the present invention there is provided the use of ZD6474 or a pharmaceutically acceptable salt thereof in the manufacture of a medicament for use in inhibiting the transformation of prostate cells into cancerous cells in a warm-blooded animal such as a human which is being treated with androgen ablation.

According to a further aspect of the present invention there is provided a therapeutic combination treatment comprising the administration of an effective amount of ZD6474 or a pharmaceutically acceptable salt thereof, optionally together with a pharmaceutically acceptable excipient or carrier, and the simultaneous, sequential or separate administration of an effective amount of an antiandrogen, wherein an antiandrogen may optionally be administered together with a pharmaceutically acceptable excipient or carrier, to a warm-blooded animal such as a human in need of such therapeutic treatment.

Such therapeutic treatment includes an antiangiogenic and/or vascular permeability effect, an anti-cancer effect and an anti-tumour effect.

Such therapeutic treatment also includes the inhibition of the transformation of cancerous cells in the prostate from a hormone-dependent state into a hormone-independent state and the inhibition of the transformation of prostate cells into cancerous cells.

A combination treatment of the present invention as defined herein may be achieved by way of the simultaneous, sequential or separate administration of the individual components of said treatment. A combination treatment as defined herein may be applied as a sole therapy or may involve additional surgery or radiotherapy or an additional chemotherapeutic agent in addition to a combination treatment of the invention.

Surgery may comprise the step of partial or complete tumour resection, prior to, during or after the administration of the combination treatment with ZD6474 described herein.

Other chemotherapeutic agents for optional use with a combination treatment of the present invention include those described in WO 01/32651 which is incorporated herein by reference. Such chemotherapy may cover five main categories of therapeutic agent:

(i) other antiangiogenic agents including vascular targeting agents;

(ii) cytostatic agents;

(iii) biological response modifiers (for example interferon);

(iv) antibodies (for example edrecolomab); and

(v) antiproliferative/antineoplastic drugs and combinations thereof, as used in medical oncology; and other categories of agent are:

(vi) antisense therapies;

(vii) gene therapy approaches; and

(ix) immunotherapy approaches.

Particular examples of chemotherapeutic agents for use with a combination treatment of the present invention are cyclophosphamide, raltitrexed, etoposide, vincristine, vinorelbine, paclitaxel, docetaxel, cisplatin, oxaliplatin, carboplatin, gemcitabine, irinotecan (CPT-11) and 5-fluorouracil (5-FU); such combinations are expected to be particularly useful for the treatment of prostate cancer.

The administration of a triple combination of ZD6474, androgen ablation and ionising radiation may produce effects, such as anti-tumour effects, greater than those achieved with any of ZD6474, androgen ablation and ionising radiation used alone, greater than those achieved with the combination of ZD6474 and androgen ablation, greater than those achieved with the combination of ZD6474 and ionising radiation, greater than those achieved with the combination of androgen ablation and ionising radiation.

According to the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of androgen ablation and before, after or simultaneously with an effective amount of ionising radiation.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of androgen ablation and before, after or simultaneously with an effective amount of ionising radiation.

In particular the cancer is prostate cancer.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of androgen ablation and before, after or simultaneously with an effective amount of ionising radiation.

In particular the cancer involving a solid tumour is prostate cancer.

According to a further aspect of the present invention there is provided a method for inhibiting the transformation of cancerous cells in the prostate from a hormone-dependent state into a hormone-independent state in a warm-blooded animal such as a human, which comprises administering to said animal an effective amount of ZD6474 or a pharmaceutically acceptable salt thereof, before, after or simultaneously with an effective amount of androgen ablation and before, after or simultaneously with an effective amount of ionising radiation.

According to a further aspect of the present invention there is provided a method for inhibiting the transformation of prostate cells into cancerous cells in a warm-blooded animal such as a human, which comprises administering to said animal an effective amount of ZD6474 or a pharmaceutically acceptable salt thereof, before, after or simultaneously with an effective amount of androgen ablation and before, after or simultaneously with an effective amount of ionising radiation.

According to a further aspect of the present invention there is provided a method for the production of an antiangiogenic and/or vascular permeability reducing effect in a warm-blooded animal such as a human, which comprises administering to said animal an effective amount of ZD6474 or a pharmaceutically acceptable salt thereof, before, after or simultaneously with an effective amount of an antiandrogen and before, after or simultaneously with an effective amount of ionising radiation, wherein ZD6474 and an antiandrogen may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of ZD6474 or a pharmaceutically acceptable salt thereof, before, after or simultaneously with an effective amount of an antiandrogen and before, after or simultaneously with an effective amount of ionising radiation, wherein ZD6474 and an antiandrogen may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of ZD6474 or a pharmaceutically acceptable salt thereof, before, after or simultaneously with an effective amount of an antiandrogen and before, after or simultaneously with an effective amount of ionising radiation, wherein ZD6474 and an antiandrogen may each optionally be administered together with a pharmaceutically acceptable excipient or carrier.

According to a further aspect of the present invention there is provided a method for for inhibiting the transformation of cancerous cells in the prostate from a hormone-dependent state into a hormone-independent state in a warm-blooded animal such as a human, which comprises administering to said animal an effective amount of ZD6474 or a pharmaceutically acceptable salt thereof, before, after or simultaneously with an effective amount of an antiandrogen and before, after or simultaneously with an effective amount of ionising radiation, wherein ZD6474 and an antiandrogen may each optionally be administered together with a pharmaceutically acceptable excipient or carrier.

According to a further aspect of the present invention there is provided a method for inhibiting the transformation of prostate cells into cancerous cells in a warm-blooded animal such as a human, which comprises administering to said animal an effective amount of ZD6474 or a pharmaceutically acceptable salt thereof, before, after or simultaneously with an effective amount of an antiandrogen and before, after or simultaneously with an effective amount of ionising radiation, wherein ZD6474 and an antiandrogen may each optionally be administered together with a pharmaceutically acceptable excipient or carrier.

According to a further aspect of the present invention there is provided the use of ZD6474 or a pharmaceutically acceptable salt thereof and an antiandrogen in the manufacture of a medicament for use in the production of an antiangiogenic and/or vascular permeability reducing effect in a warm-blooded animal such as a human which is being treated with ionising radiation.

According to a further aspect of the present invention there is provided the use of ZD6474 or a pharmaceutically acceptable salt thereof and an antiandrogen in the manufacture of a medicament for use in the production of an anti-cancer effect in a warm-blooded animal such as a human which is being treated with ionising radiation.

In particular the cancer is prostate cancer.

According to a further aspect of the present invention there is provided the use of ZD6474 or a pharmaceutically acceptable salt thereof and an antiandrogen in the manufacture of a medicament for use in the production of an anti-tumour effect in a warm-blooded animal such as a human which is being treated with ionising radiation.

In particular the tumour is a prostate cancer tumour.

According to a further aspect of the present invention there is provided the use of ZD6474 or a pharmaceutically acceptable salt thereof and an antiandrogen in the manufacture of a medicament for use in inhibiting the transformation of cancerous cells in the prostate from a hormone-dependent state into a hormone-independent state in a warm-blooded animal such as a human which is being treated with ionising radiation.

According to a further aspect of the present invention there is provided the use of ZD6474 or a pharmaceutically acceptable salt thereof and an antiandrogen in the manufacture of a medicament for use in inhibiting the transformation of prostate cells into cancerous cells in a warm-blooded animal such as a human which is being treated with ionising radiation.

According to a further aspect of the present invention there is provided the use of ZD6474 or a pharmaceutically acceptable salt thereof in the manufacture of a medicament for use in the production of an antiangiogenic and/or vascular permeability reducing effect in a warm-blooded animal such as a human which is being treated with androgen ablation and which is being treated with ionising radiation.

According to a further aspect of the present invention there is provided the use of ZD6474 or a pharmaceutically acceptable salt thereof in the manufacture of a medicament for use in the production of an anti-cancer effect in a warm-blooded animal such as a human which is being treated with androgen ablation and which is being treated with ionising radiation.

In particular the cancer is prostate cancer.

According to a further aspect of the present invention there is provided the use of ZD6474 or a pharmaceutically acceptable salt thereof in the manufacture of a medicament for use in the production of an anti-tumour effect in a warm-blooded animal such as a human which is being treated with androgen ablation and which is being treated with ionising radiation.

In particular the tumour is a prostate cancer tumour.

According to a further aspect of the present invention there is provided the use of ZD6474 or a pharmaceutically acceptable salt thereof in the manufacture of a medicament for use in inhibiting the transformation of cancerous cells in the prostate from a hormone-dependent state into a hormone-independent state in a warm-blooded animal such as a human which is being treated with androgen ablation and which is being treated with ionising radiation.

According to a further aspect of the present invention there is provided the use of ZD6474 or a pharmaceutically acceptable salt thereof in the manufacture of a medicament for use in inhibiting the transformation of prostate cells into cancerous cells in a warm-blooded animal such as a human which is being treated with androgen ablation and which is being treated with ionising radiation.

According to a further aspect of the present invention there is provided a therapeutic combination treatment comprising the administration of an effective amount of ZD6474 or a pharmaceutically acceptable salt thereof, optionally together with a pharmaceutically acceptable excipient or carrier, and the administration of an effective amount of an antiandrogen, optionally together with a pharmaceutically acceptable excipient or carrier and the administration of an effective amount of ionising radiation, to a warm-blooded animal such as a human in need of such therapeutic treatment wherein the ZD6474, antiandrogen and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising ZD6474 and androgen ablation. For example said ionising radiation may be given to said warm-blooded animal such as a human within the period of a week before to a week after the administration of a medicament or combination treatment comprising ZD6474 and androgen ablation. This means that ZD6474, androgen ablation and ionising radiation may be administered separately or sequentially in any order, or may be administered simultaneously. The warm-blooded animal may experience the effect of each of ZD6474, androgen ablation and radiation simultaneously.

According to one aspect of the present invention the ionising radiation is administered before one of ZD6474 and androgen ablation or after one of ZD6474 and androgen ablation.

According to one aspect of the present invention the ionising radiation is administered before both ZD6474 and androgen ablation or after both ZD6474 and androgen ablation.

According to one aspect of the present invention ZD6474 is administered to a warm-blooded animal after the animal has been treated with ionising radiation. According to another aspect of the present invention the effect of a method of treatment of the present invention is expected to be at least equivalent to the addition of the effects of each of the components of said treatment used alone, that is, of each of ZD6474 and androgen ablation used alone or of each of ZD6474, androgen ablation and ionising radiation used alone.

According to another aspect of the present invention the effect of a method of treatment of the present invention is expected to be greater than the addition of the effects of each of the components of said treatment used alone, that is, of each of ZD6474 and androgen ablation used alone or of each of ZD6474, androgen ablation and ionising radiation used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with ZD6474 or androgen ablation or ionising radiation alone. Further, the effect of the combination treatment is synergistic if a beneficial effect is obtained in a group of patients that does not respond (or responds poorly) to ZD6474 or androgen ablation or ionising radiation alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of ZD6474 or androgen ablation or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

As stated above the combination treatments of the present invention as defined herein are of interest for their antiangiogenic and/or vascular permeability effects. Angiogenesis and/or an increase in vascular permeability is present in a wide range of disease states including cancer (including leukemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, lymphoedema, endometriosis, dysfunctional uterine bleeding and ocular diseases with retinal vessel proliferation including age-related macular degeneration. Combination treatments of the present invention are expected to be particularly useful in the prophylaxis and treatment of prostate cancer. Combination treatments of the present invention may also be useful in the prophylaxis and treatment of benign diseases of the prostate such as benign prostatic hypertrophy/benign prostatic hyperplasia (BPH).

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the ZD6474 of the combination treatment may be delivered endoscopically, intratracheally, intralesionally, percutaneously, intravenously, subcutaneously, intraperitoneally or intratumourally. Preferably ZD6474 is administered orally. In general the compositions described herein may be prepared in a conventional manner using conventional excipients. The compositions of the present invention are advantageously presented in unit dosage form.

ZD6474 will normally be administered to a warm-blooded animal at a unit dose within the range 10-500 mg per square metre body area of the animal, for example approximately 0.3-15 mg/kg in a human. A unit dose in the range, for example, 0.3-15 mg/kg, preferably 0.5-5 mg/kg is envisaged and this is normally a therapeutically-effective dose. A unit dosage form such as a tablet or capsule will usually contain, for example 25-500 mg of active ingredient. Preferably a daily dose in the range of 0.5-5 mg/kg is employed.

Antiandrogens may be dosed according to known routes of administration and dosages. For example bicalutamide may be dosed at 150 mg per day as a single daily oral dose. For example goserelin may be administered by subcutaneous injection either 3.6 mg every 28 days or 10.8 mg every 12 weeks.

The dosages and schedules may vary according to the particular disease state and the overall condition of the patient. Dosages and schedules may also vary if, in addition to a combination treatment of the present invention, one or more additional chemotherapeutic agents is/are used. Scheduling can be determined by the practitioner who is treating any particular patient.

Radiotherapy may be administered according to the known practices in clinical radiotherapy. The dosages of ionising radiation will be those known for use in clinical radiotherapy. The radiation therapy used will include for example the use of y-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

As stated above the size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

The present invention relates to combinations of androgen ablation with ZD6474 or with a salt of ZD6474.

Salts of ZD6474 for use in pharmaceutical compositions will be pharmaceutically acceptable salts, but other salts may be useful in the production of ZD6474 and its pharmaceutically acceptable salts. Such salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

ZD6474 may be synthesised according to any of the known processes for making ZD6474. For example ZD6474 may be made according to any of the processes described in WO 01/32651; for example those described in Examples 2(a), 2(b) and 2(c) of WO 01/32651.

Anti-androgens are commercially available.

A preferred antiandrogen is bicalutamide.

A preferred antiandrogen is goserelin.

The following test may be used to demonstrate the activity of ZD6474 in combination with androgen ablation.

### LNCaP Human Prostate Cancer Xenograft Model

Six to eight-week-old Swiss nude mice were given one injection in the anterior flank with 5×10⁶ tumour cells suspended in 0.1 ml of serum-free medium with the addition of 100 μl of Matrigel. Once tumours were established, mice were randomized into control and treatment groups. ZD6474 was suspended in a 1% (v/v) solution of polyoxyethylene (20) sorbitan mono-oleate in deionized water and administered by daily oral gavage at 0.1 ml/10 g body weight.

The impact of ZD6474 on tumour growth was examined at a dose of 50 mg/kg/day in both tumour-bearing normal and castrated mice versus appropriate control groups.

Mice were examined twice a week, and tumours were measured with calipers across the greatest two diameters. At the time of euthanasia, tumours were removed by dissection away from adjacent organs and structures and weighed on an analytical balance. For histologic analysis, tissue samples were fixed in 10% neutral buffered formalin and processed through graded ethanols and xylenes for paraffin embedding and staining using standard methods. For calculation of percent necrosis, 5 random fields per tumour were examined at low power (10×) from tumours harvested at 40 days. The necrotic area(s) in each field was/were circumscribed electronically using the tools available in the ImagePro computerized image analysis package (Media Cybernetics) to create an area. A human operator carried out this identification and circling of the necrotic area for all analyzed issues. Using the total area of the section as the denominator, the percentage of necrosis was evaluated for each field. To obtain an average for a whole experimental tumour group, the percent necrosis for each field was averaged across the total number of tumours in the group.

Repeated measure models were used for the analysis of the in vivo tumour volumes. Several covariance structures, including AR- 1 and random coefficient models, were used and all yielded similar conclusions. The analyses reported herein are based on a random coefficient model with robust estimates of the covariance matrix. Specific comparisons between treatment groups were made using F-test based on contrasts. Statistical analyses were carried out in SAS PROC MIXED; the plots were prepared in GAUSS 5.0. Error bars represent one standard error from the mean. Time from treatment initiation to the time that tumour volumes exceeded a cut-off value (a tumour volume of >300 mm³) were estimated with Kaplan-Meier curves and compared across treatment groups with the log-rank test. Tumour volume cut-offs were used to avoid using a survival endpoint, which would have involved significant animal distress and to employ a threshold that was appropriate for comparing the tumour growth data across all groups.

At the doses used the effects on LNCaP xenograft growth in vivo associated with 50 mg/kg/day of ZD6474, are most likely to be related to an effect on VEGF receptor signaling.

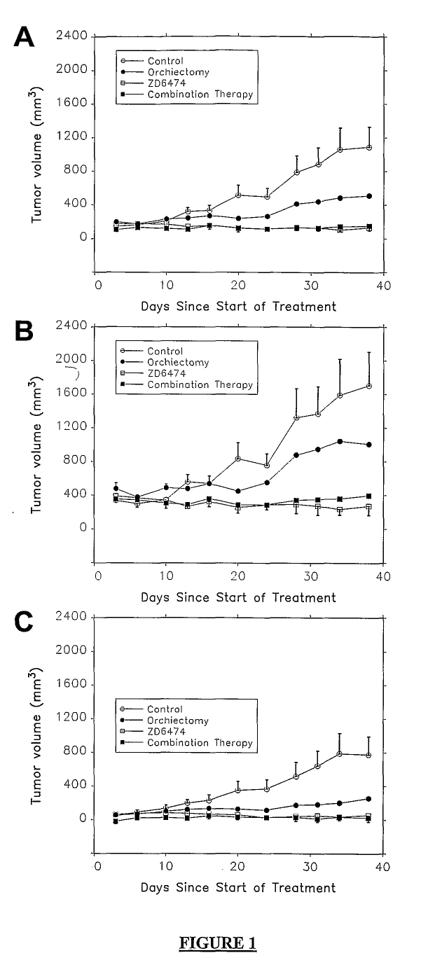

Xenografts reached an average volume of 134 mm³ (range 4-616 mm³) before treatment. There were no significant differences in tumour volume among the groups at the start of treatment. After 38 days of ZD6474 treatment significant differences in tumour volume were observed between the ZD6474 group and the orchiectomy (P<0.001) and control groups (P<0.001) (FIG. 1A). Similarly, significant differences were observed between the combination therapy group and the orchiectomy (P<0.001) and control groups (P<0.001).

Statistically significant differences (P<0.05) were found between the ZD6474 treatment group and the orchiectomy group for days 13 to 38 post-treatment. This result may suggest that ZD6474 maintains tumour stasis while tumours in the mice that have undergone androgen ablation become androgen refractory.

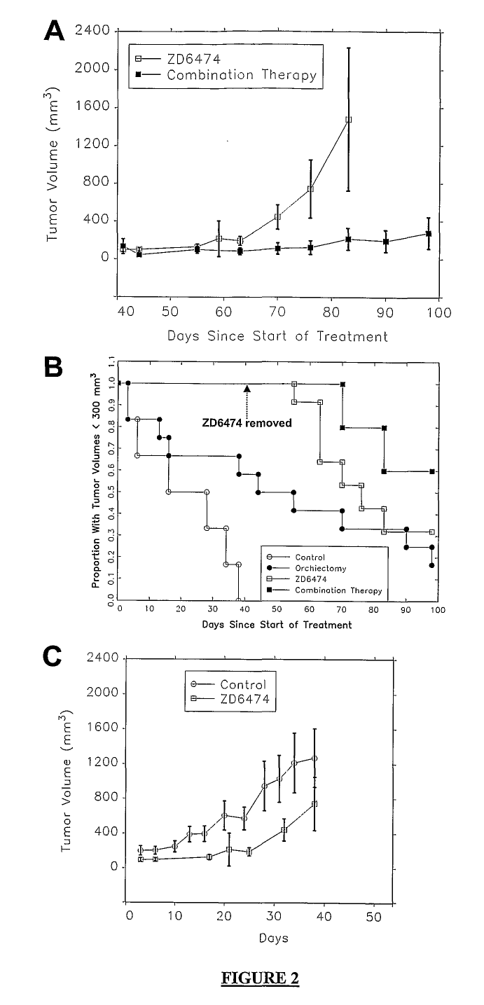

In animals treated with ZD6474 alone for 40 days and then monitored following compound withdrawal, tumour growth resumed after a delay of a few days (˜15 days) (FIG. 2A). The delay is also evident from the Kaplan Meier analysis (FIG. 2B). The delay in tumour regrowth may in part be due to the time needed to remove compound from tissues and/or an overestimate of viable tumour tissue from caliper measurements, since chronic administration of ZD6474 can induce significant tumour necrosis. Careful examination of the rate of tumour growth of untreated and previously ZD6474 treated tumours suggested there was no significant difference between the two, when the delay of approximately 15 days is taken in to account (FIG. 2C).

When ZD6474 removal was examined in orchiectomised mice, the combination of androgen ablation with a fixed period of ZD6474 treatment was found to produce the greatest net therapeutic effect (FIG. 2A). Evidence of tumour growth, relative to the tumour volumes measured on the day of ZD6474 withdrawal, was only apparent 40-58 days after compound removal. The data suggests that the cytostatic effect of androgen ablation is maintained even after the discontinuation of ZD6474 in animals that were treated with both modalities.

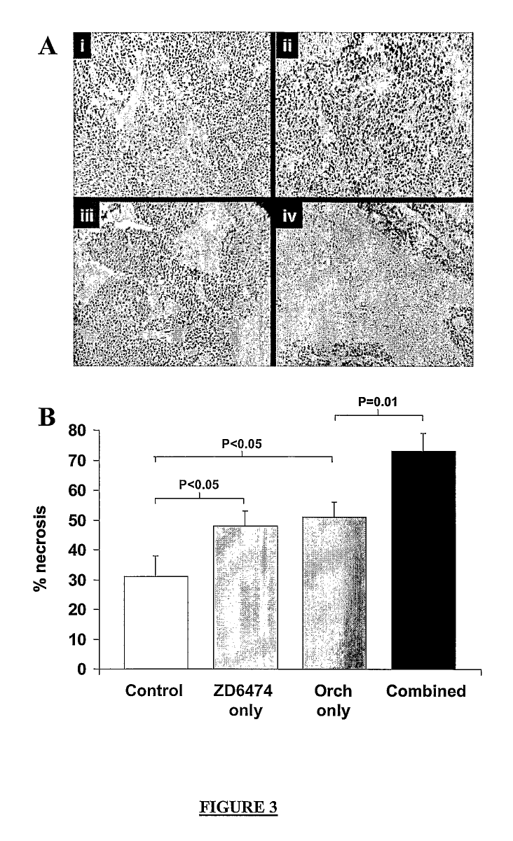

To examine the consequences of treatment by immunohistology we evaluated 5 tumours from each experimental group at day 40 after initiation of treatment. Tumour xenografts from mice treated with 50 mg/kg/day ZD6474 (once daily, p.o.), or from those undergoing orchiectomy, were found to have a higher percentage of necrosis when compared with vehicle-treated controls (48±5% and 51.1±5% versus 31±7% respectively, P=0.047) (FIG. 3). Animals that were treated with the combination of orchiectomy and ZD6474 had tumours with more necrosis (73±6% versus 51±5%, P=0.01) than was observed in either monotherapy group.

When ZD6474 treatment was withdrawn, the greatest inhibition of tumour re-growth was observed in androgen-ablated animals. Increased tumour necrosis was also observed in mice treated with the combination of these two approaches.

Analogous experiments may be used to look at the combinations of ZD6474 with an antiandrogen and ZD6474 and androgen ablation with ionising radiation.

The data are presented graphically in FIGS. 1, 2 and 3.
FIG. 1: Effect of vehicle (control), ZD6474 (50 mg/kg/day), orchiectomy or ZD6474 (50 mg/kg/day) and orchiectomy on the growth of LNCaP tumour xenografts. Xenografts were established subcutaneously in nude mice and reached an average (all treatment groups combined) volume of 134 mm³ (range 4-616 mm³) before treatment. Once-daily oral administration of ZD6474 or vehicle was then started and continued for the duration of the experiment. Data points represent a mean from nine mice in the control group, 12 mice in the orchiectomy group, 15 mice in the ZD6474 group and 12 mice in the combination therapy group, with SEs shown in one direction. Castration was carried out at the same time as initiation of ZD6474. Data represent tumour size (mm³) after subtracting the last pre-treatment tumour volume within each animal. A, Estimated tumour growth among groups at median initial tumour volume (134 mm³). B, Estimated tumour growth among groups at 90th percentile of tumour volume (339 mm³). C, Estimated tumour growth among groups at 25th percentile of tumour volume (30.4 mm³).
FIG. 2: Effect of ZD6474 (50 mg/kg/day) discontinuation on the growth of LNCaP tumour xenografts. At day 40, chronic treatment with ZD6474 was discontinued and tumour monitoring continued. A, Data points (± SE) represent a mean from 13 normal mice and 6 mice that were orchiectomised prior to receiving ZD6474. Data represent changes in tumour size (mm³). The groups were statistically different (P<0.001, F-test). Error bars indicate one standard error of the mean. B, Kaplan-Meier estimates of the time until tumour volumes reached 300 mm³. Animals whose tumours had reached 300 mm³ before treatment initiation were excluded. Animals euthanized with tumour volume less than 300 mm³ were censored at the last follow-up time. C, Comparative growth plot of tumours in Control and ZD6474 pretreated (i.e. after drug discontinuation) mice (days 0-35 for control group and days 41-76 for ZD6474 group). Data are means from 9 animals in the control group and 13 animals in the ZD6474 group.
FIG. 3: histological analysis of 5 LNCaP tumour xenografts treated for 40 days. A, Hematoxylin and eosin histological sections (10× objective) picked at random from (i) control (vehicle-treated); (ii) Orchiectomy; (iii) ZD6474 (50 mg/kg/day)-treated; (iv) Orchiectomy+ZD6474 (50 mg/kg/day)-treated. All tumours were harvested at 40 days. A significant increase in the percentage of total tumour necrosis was noted in the ZD6474 (50 mg/kg/day, once daily, p.o.) plus orchiectomy group, compared with tumours from mice receiving either treatment alone. B, Percentage of tumour necrosis as determined by morphometric image analysis of tumours described in A.

1-10. (canceled)

11. A pharmaceutical composition which comprises ZD6474 or a pharmaceutically acceptable salt thereof, and an antiandrogen, in association with a pharmaceutically acceptable excipient or carrier.

12. A kit comprising ZD6474 or a pharmaceutically acceptable salt thereof, and an antiandrogen.

13-22. (canceled)

23. A method for the production of an antiangiogenic and/or vascular permeability reducing effect in a warm-blooded animal, which comprises administering to said animal an effective amount of ZD6474 or a pharmaceutically acceptable salt thereof, before, after or simultaneously with an effective amount of androgen ablation.

24. A method for the production of an antiangiogenic and/or vascular permeability reducing effect in a warm-blooded animal, which comprises administering to said animal an effective amount of ZD6474 or a pharmaceutically acceptable salt thereof, before, after or simultaneously with an effective amount of androgen ablation and before, after or simultaneously with an effective amount of ionising radiation.

25. The composition according to claim 11 wherein the antiandrogen is selected from a luteinising hormone releasing hormone agonist, a luteinising hormone releasing hormone antagonists, a non-steroidal antiandrogen and a steroidal antiandrogen.

26. The composition according to claim 11 wherein the antiandrogen is selected from goserelin, buserelin, triptorelin, leuprorelin, bicalutamide (or an enantiomer thereof), flutamide, nilutamide, cyproterone acetate and megestrol acetate.

27. The composition according to claim 11 wherein the antiandrogen is bicalutamide (or an enantiomer thereof).

28. The composition according to claim 11 wherein the antiandrogen is goserelin.

29. The method according to claim 23 or claim 24 wherein the androgen ablation is achieved by surgical means.

30. The method according to claim 23 or claim 24 wherein the androgen ablation is achieved by chemical means.

31. The method according to claim 30 wherein the chemical means is an antiandrogen selected from a luteinising hormone releasing hormone agonist, a luteinising hormone releasing hormone antagonists, a non-steroidal antiandrogen and a steroidal antiandrogen.

32. The method according to claim 30 wherein the chemical means is an antiandrogen selected from goserelin, buserelin, triptorelin, leuprorelin, bicalutamide (or an enantiomer thereon, flutamide, nilutamide, cyproterone acetate and megestrol acetate.

33. The method according to claim 30 wherein the chemical means is bicalutamide (or an enantiomer thereof).

34. The method according to claim 30 wherein the chemical means is goserelin.

35. The method according to claim 23 or claim 24 wherein the warm-blooded animal is a human with prostate cancer.

36. The method according to claim 24 which further comprises the administration of a chemotherapeutic agent.

37. The method according to claim 30 which further comprises the administration of a chemotherapeutic agent.

38. The method according to claim 36 wherein the chemotherapeutic agent is selected from cyclophosphamide, raltitrexed, etoposide, vincristine, vinorelbine, paclitaxel, docetaxel, cisplatin, oxaliplatin, carboplatin, gemcitabine, irinotecan (CPT-11) and 5-fluorouracil (5-FU).

39. The method according to claim 37 wherein the chemotherapeutic agent is selected from cyclophosphamide, raltitrexed, etoposide, vincristine, vinorelbine, paclitaxel, docetaxel, cisplatin, oxaliplatin, carboplatin, gemcitabine, irinotecan (CPT-11) and 5-fluorouracil (5-FU).
